(19) United States
(12) Patent Application Publication
(10) Pub. No.: US 2016/0083658 A1
(43) Pub. Date: Mar. 24, 2016

(54) METHODS FOR PRODUCTION OF LIQUID HYDROCARBONS FROM ENERGY, CO2 AND H2O

(71) Applicant: Gunnar SANNER, Stathelle (NO)

(72) Inventor: Gunnar SANNER, Stathelle (NO)

(21) Appl. No.: 14/785,088

(22) PCT Filed: Apr. 10, 2014
(86) PCT No.: PCT/EP2014/057267

§ 371 Date: Oct. 16, 2015

(30) Foreign Application Priority Data
Apr. 19, 2013 (NO) 20130543

(51) Int. Cl. C10G 2/00; C07C 29/151; C01B 3/12

(52) U.S. Cl. CPC C10G 2/30; C01B 3/12; C07C 29/1518; C01B 2203/0216; C01B 2203/062

(57) ABSTRACT

Energy uploading method transferring energy into liquid hydrocarbon comprising the steps a) preparing a mixture of hydrogen and carbon monoxide from carbon dioxide, H₂O and energy, b) reacting said mixture to form liquid hydrocarbon, c) transferring heat energy from the formed liquid hydrocarbon to the carbon dioxide and or the H₂O.

The present invention relates to methods for production of liquid hydrocarbons from energy, CO₂ and H₂O. Especially the present invention relates to integrated and energy efficient methods for transforming energy, CO₂ and H₂O to liquid hydrocarbons applicable for use as fuel or for other purposes.

### BACKGROUND

The transformation of renewable energy, H₂O and CO₂ to liquid hydrocarbons could be named Renewable (energy)-to-Liquid (RTL). The main purpose of these reactions is as the name indicates to transform energy, as power and/or heat, to hydrocarbons that are liquid at room temperature and near atmospheric pressure. Not to limit the energy source to renewable energy, but include any form of power or heat input, and not to limit the end products to liquid, but include all types of hydrocarbons, we further use the term “Energy upload”. The produced liquid hydrocarbons are compact energy carriers, easy to handle and applicable as raw materials for other processes such as production of polymers. In addition to hydrocarbons, the solutions also produce substantial amounts of O₂, a gas useful for industrial purposes, e.g.: GTL, metal industry, oxyfuel power plants.

Different processes are known for performing Energy upload today. The main principle of the existing Energy upload plants is decomposition of water to form hydrogen and oxygen gas, and thereafter combine hydrogen with CO₂ to form hydrocarbons.

### PRIOR ART

An energy system for connecting Energy Upload plants with corresponding energy Offload plants and form a closed energy system are disclosed in WO2012/069635 and WO2012/069636.

US2012/0228150 discloses the processing of syngas into synthetic liquid fuel in the form of alkanes. Hydrogen for the syngas is produced by electrolysis of water. Also disclosed is the production of methanol from hydrogen and carbon monoxide, where the hydrogen is obtained from thermal pyrolysis of methane.

US2012/0259025 discloses the formation of gaseous methane from hydrogen and carbon dioxide in a Sabatier reactor. The hydrogen is obtained through water electrolyses.

### Objectives of the Invention

The objective of the present invention is to provide an integrated method for transforming energy as power and/or heat, CO₂ and H₂O to liquid hydrocarbons.

A further objective is to provide a method with increased cost efficiency and increased energy efficiency of the process.

Yet another objective of the present invention is to provide a method which can be performed with thermal energy as the additional energy input, more preferably with sustainable energy as the additional energy input.

The goal is to produce alkanes and alcohols in liquid form at standard conditions (e.g.: 20 or 25° C. and 1 atmosphere pressure). Hydrocarbons in liquid form are more valuable and transportable than hydrocarbons in gaseous form. Hydrocarbons in liquid form can be transported in ships and stored without use of pressure- and/or cooled tanks. In the consumer markets liquid hydrocarbons are used to a large extend in transportation sectors like cars, trucks, ships and planes. Liquid hydrocarbons are presently the highest priced energy products per energy unit.

It is an aim to provide an energy efficient process. The present invention provides an energy uploading method transferring energy into liquid hydrocarbon comprising steps

a) preparing a mixture of hydrogen and carbon monoxide from carbon dioxide, H₂O and energy,

b) reacting said mixture to form liquid hydrocarbon,

c) transferring heat energy from the formed liquid hydrocarbon to the carbon dioxide and or the H₂O.

In one aspect of the present invention the step a) comprises decomposition of carbon dioxide into carbon monoxide and oxygen.

In a further aspect step a) further comprises reacting a part of the carbon monoxide with H₂O to form carbon dioxide and hydrogen and transferring the formed carbon dioxide to the decomposition of carbon dioxide.

In yet a further aspect the step a) comprises decomposition of water into oxygen and hydrogen.

In another aspect of the present invention the step a) comprises combined steam reforming and carbon dioxide reforming

The present invention also provides an energy uploading method transferring energy into liquid hydrocarbon comprising steps

d) preparing hydrogen from H₂O and energy,

e) preparing a mixture of hydrogen and carbon dioxide,

f) reacting said mixture to form liquid hydrocarbon,

g) transferring heat energy from the formed liquid hydrocarbon to the carbon dioxide and or the H₂O.

According to a further aspect of any of the methods according to the present invention oxygen is produced as a by-product. In a further aspect the method comprises transferring heat from the oxygen to the carbon dioxide and or the H₂O.

In one aspect of the present invention the energy supplied is heat energy, and in an aspect thereof the energy supplied is sustainable energy.

In one aspect of the methods the liquid hydrocarbon is alcohol CₙH₂ₙ₊₁OH, where n=1-20, preferably n=1-6.

In another aspect of the methods the liquid hydrocarbon is alkane CₙH₂ₙ₊₂, where n=5-17, preferably n=5-10.

In yet another aspect of the methods according to the present invention, wherein the step c) or g) comprises converting heat from exothermic reactions to power to be used in endothermic processes

The term “liquid” in connection with hydrocarbons, alkanes and alcohols as used herein refers to phase condition of the hydrocarbon at near atmospheric conditions. For alkanes the number of carbon atoms within the compound being between 5 and 17 which is equivalent to the number of carbon atoms being higher than or equal to five for the alkane to be described as liquid, whereas for alcohols also compounds with only one carbon atom such as methanol falls within the term liquid, typically alcohols are n=1-5. The method could also be used to produce gas alkanes (n=1,2,3,4) or solid alkanes where n>=18.

The source of the carbon dioxide for the method can be any known CO₂ source such as CO₂ from reservoirs, CO₂ captured from industry or CO₂ captured from air, or combinations thereof.

Thermal energy can be utilized as energy input. In an attractive embodiment sustainable energy is employed as the sole or main energy input, e.g.: solar thermal, geothermal. Other thermal energy sources could also be used like nuclear; electricity input is also an option. Applicable energy sources also include other type of energy (bio or fossil fuel)

The main principals of the present invention may be employed in the production of alkanes, alcohols and other liquid hydro carbons. The total reaction schemes for alkanes is

(n)CO₂+(n+1)H₂O=>CₙH₂ₙ₊₂+(3n+1)/2O₂,

wherein n=alkane number

The total reaction schemes for alcohols is

(n)CO₂+(n+1)H₂O=>CₙH₂ₙ₊₁OH+(3/2)nO₂,

wherein n=alcohol number.

Examples of specific total reactions are:

16CO₂+18H₂O=>2C₈H₁₈+25O₂ (Octane)

2CO₂+3H₂O=>C₂H₅OH+3O₂ (Ethanol)

2CO₂+4H₂O=>2CH₃OH+3O₂ (Methanol)

One or more of the following advantages can be obtained by the present invention:
The present invention would be CO₂-neutral since as much CO₂ is bound in the process as is released when the fuel is burned, if renewable power/heat is used as energy input and CO₂ used is captured from industry or from air.
Combustion of the obtained liquid hydrocarbon as fuel will have less CO₂-footprint than crude oil based fuels due to no CO₂ footprint in the production process, if renewable power/heat is used as energy input and CO₂ used is captured from industry or from air.
The process could utilize CO₂ from reservoirs, CO₂ captured from industry or CO₂ captured from air.
The present solution may utilize heat as energy input, and thereby lower the cost of the energy needed to run the process.
The present invention could be used as a renewable energy or nuclear energy storage and/or energy export route. Periodically over-supply of renewable energy or nuclear energy can by this method be utilized to convert H₂O and CO₂ to liquid fuels; hence the renewable energy would be exported as “Renewable hydrocarbons”, CO₂-neutral liquid hydrocarbon fuels.

In an aspect of the present invention the processes of the alkane production and the alcohol production may be combined so that a combination of liquid alkanes and alcohols are obtained from energy, H₂O and carbon dioxide.

### BRIEF DESCRIPTION OF THE DRAWINGS

The present invention will be exemplified in further detail with reference to the enclosed figures.

FIG. 1 illustrates a first embodiment for alkane production.

FIG. 2 illustrates a second embodiment for alcohol production.

FIG. 3 illustrates an alternative third embodiment for alkane production.

FIG. 4 illustrates a fourth embodiment for alcohol production.

FIG. 5 illustrates an alternative fifth embodiment for alkane production.

FIG. 6 illustrates a sixth embodiment for alcohol production.

FIG. 7 is a schematically illustration of the main principal of the present invention.

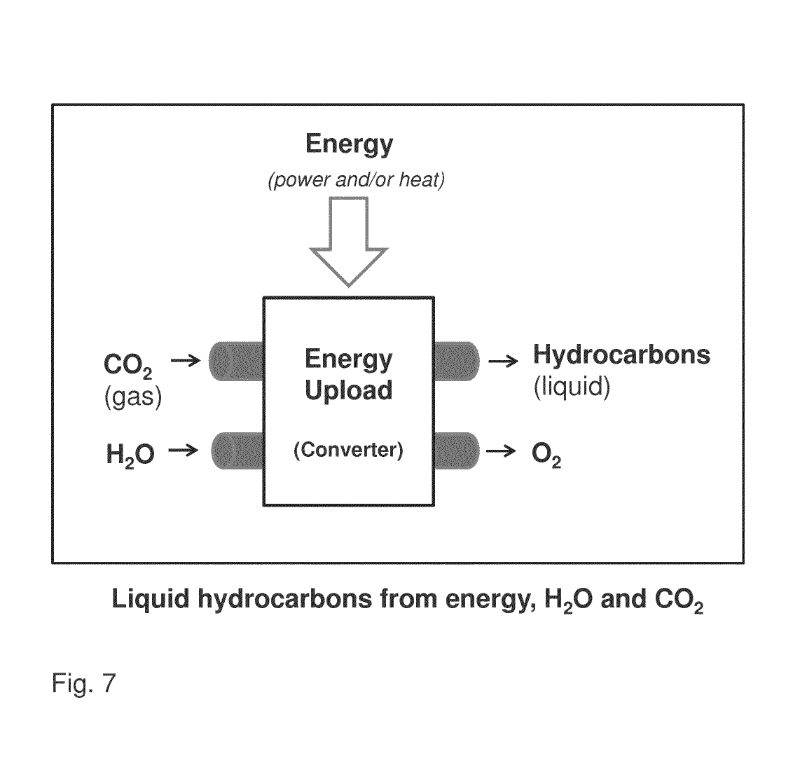

FIG. 8 illustrates the transfer of heat between inlet streams and product streams.

### PRINCIPAL DESCRIPTION OF THE INVENTION

The main concept of the present invention is illustrated on FIG. 9. The present invention provides an integrated solution to convert energy as power and/or heat to liquid fuels. In the known process of Energy Upload endothermic reactions are being employed or considered employed for the storage of renewable energy sources such as geothermal heat, sun light or wind energy resources. The renewable- or other types of energy provide the energy to react H₂O with CO₂ to form hydrocarbons. This illustration is a simplification as the process is normally performed as at least a two step process, wherein power is supplied in electrolysis of water to form hydrogen and oxygen and then in a second step the hydrogen is reacted with carbon dioxide to form hydrocarbons. The combined process has the potential of utilizing heat as renewable energy input and thereby provides a more cost efficient process, since heat usually has lower cost per energy unit than power

The input energy is transmitted into the solutions as heat or power. This energy shall be used for the chemical reactions purposes. Rest heat in the in the produced outflowing chemicals (alkanes/alcohols, and O₂) may advantageously be reclaimed by heat exchange systems. This heat is transferred into the inflowing chemicals (CO₂ and H₂O). To secure limited energy leakage, insulation will be provided around all processes with high temperature. This construction will make the solutions very energy efficient. By this the input energy will efficiently be used to fill the gap between the high chemical energy potential in produced alkanes or alcohols and the low chemical energy potential in the inflowing CO₂ and H₂O.

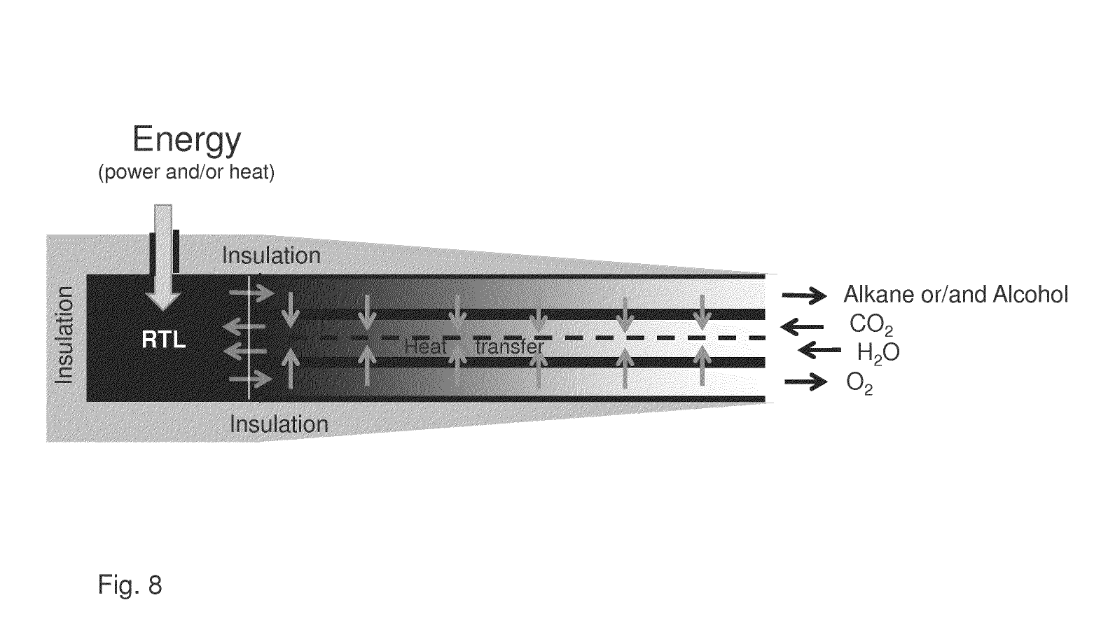

FIG. 8 shows one set up of such combination of insulation and heat transfer from outflowing to inflowing chemicals. Pipe-in-pipe solution with countercurrent flow ensures a heat gradient that allows heat to go from outflowing chemicals to inflowing. Theoretically if all heat is regained the energy input needed or the heat energy produced is determined by the energy produced and released by the chemical reactions.

The following table shows the overall difference in enthalpy for three examples of Energy Upload reactions according to the present invention.

| EXAMPLE | OCTANE | ETHANOL | METHANOL |
|---|---|---|---|
| REACTIONS | (Alkane, n = 8) | (Alcohol, n = 2) | (Alcohol, n = 1) |
| Energy Upload | 16 CO₂ + 18 | 2 CO₂ + 3 | 2 CO₂ + |
| | H₂O => 2 | H₂O => | 4 H₂O => |
| | C₈H₁₈ + 25 O₂ | C₂H₅OH + 3 O₂ | 2 CH₃OH + 3 O₂ |
| ΔH (kJ/mole) − | 5471 | 1367 | 727 |
| per mole | | | |
| HC molecule | | | |

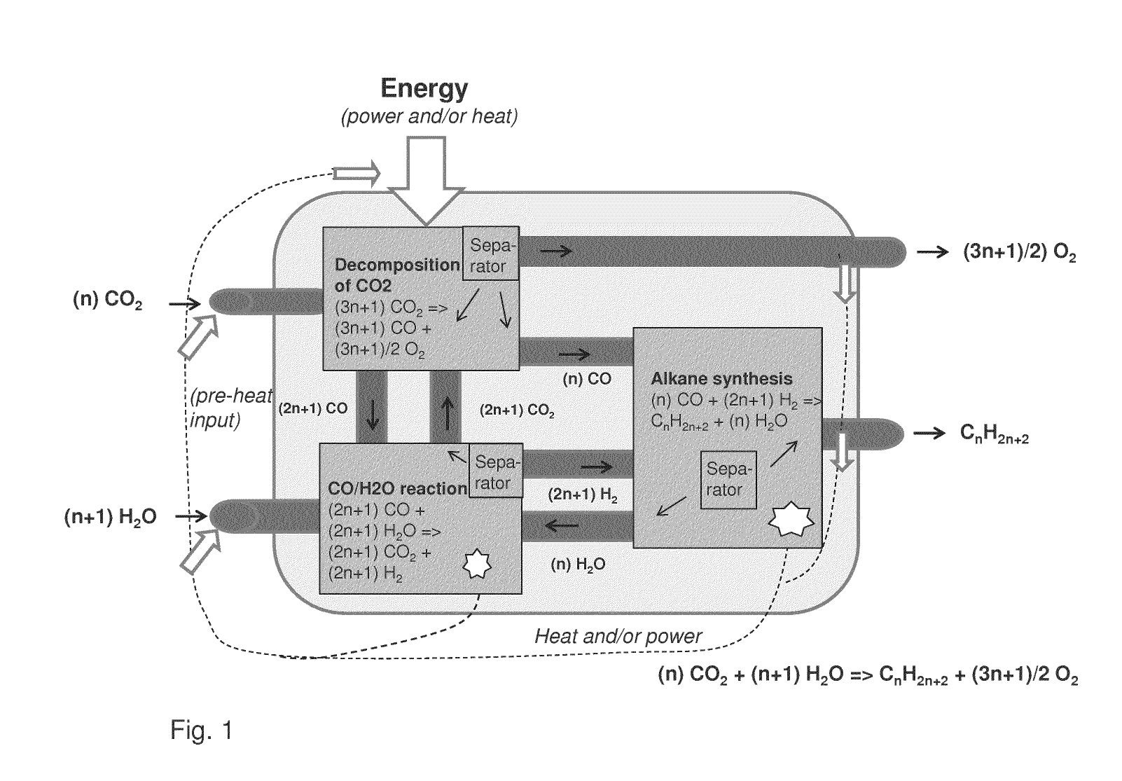

The energy efficiency of the conversion is enforced by insulation around the converter and heat transfer from outflowing products to inflowing material streams, by use of countercurrent pipe-in-pipe system as illustrated in FIG. 8, or any other methods of transferring heat. Heat based power generation could be built as part of this heat transfer from warm to cold product streams. This power generation is marked as star in the exothermic processes in the FIGS. 1 to 6. This power could be used as input to endothermic processes.

FIG. 1 illustrates a first embodiment of the present invention for the production of alkanes. Here the process is split into three reactions, decomposition of CO₂, CO/H₂O reaction and alkane synthesis. Each of these steps are in them self known processes but the integrated combination as disclosed is new.

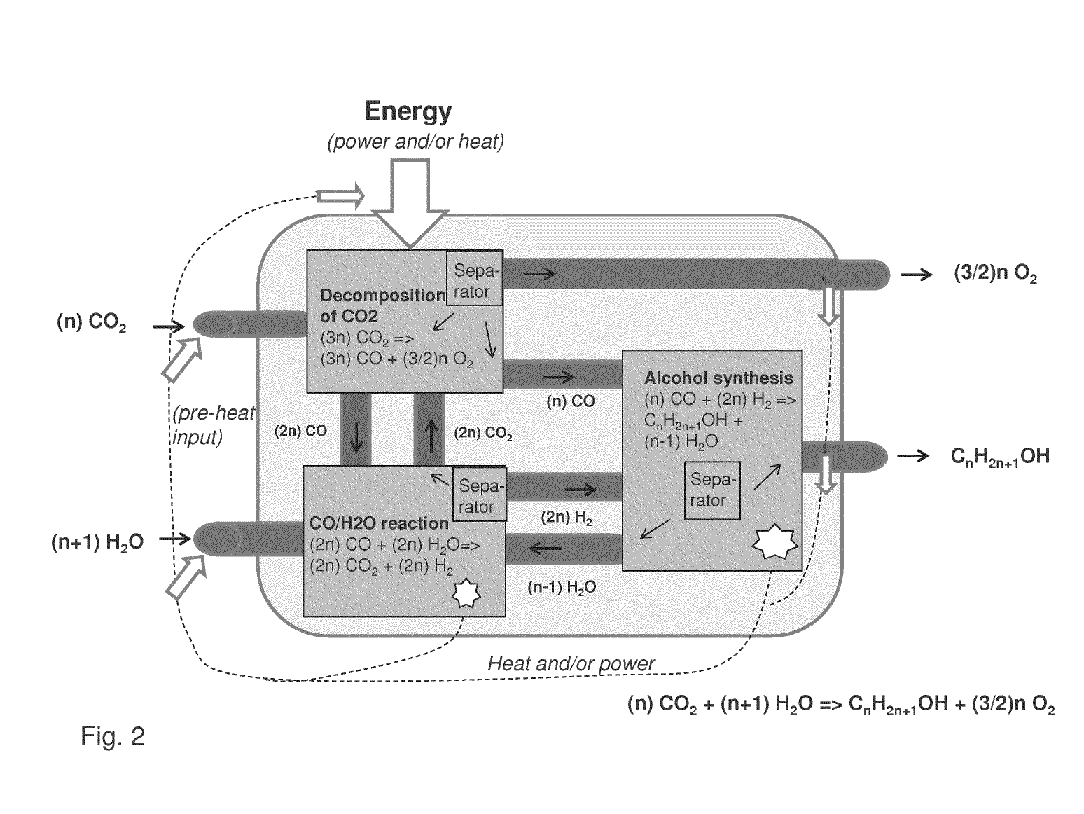

In the decomposition of CO₂ process CO₂ is split into CO and O₂ with energy as input. Some of the CO is led into CO/H₂O reaction where it is transformed to H₂ and CO₂. The produced CO₂ is led back to the decomposition of CO₂ while H₂ is led into the alkane synthesis. The alkane synthesis also receives some CO from the decomposition of CO₂ process. Water produced in the alkane synthesis process is led back to the CO/H₂O reaction. The named processes can be performed at different conditions and the present invention is not limited to any of these known methods. Taken as a whole the inlet streams are H₂O and CO₂ and the outlet streams are liquid alkanes CₙH₂ₙ₊₂ where n=5-17 and O₂. The energy consumption and production is also illustrated in FIG. 2 by the fat arrows and the stars. Energy is added to the CO₂ decomposition process to provide the heat for the process. This heat can at least partly be supplied by pre-heating the CO₂ with surplus of energy from the exothermic alkane synthesis or CO/H₂O reaction.

FIG. 2 illustrate a second embodiment of the present invention which differs from the embodiment of FIG. 2 only in that the alkane synthesis is replaced with an alcohol synthesis, so liquid hydrocarbon formed by the overall process is an alcohol C₁₁H₂ₙ₊₁OH, where n>=1, preferably n=1-20, more preferably n=1-10.

The total reactions of embodiment 1 and 2:

Alkane Production (1):

(3n+1)CO₂=>(3n+1)CO+(3n+1)/2O₂

(2n+1)CO+(2n+1)H₂O=>(2n+1)CO₂+(2n+1)H₂

(n)CO+(2n+1)H₂=>CₙH₂ₙ₊₂+nH₂O

Alcohol Production (2):

(3n)CO₂=>(3n)CO+(3/2)nO₂

(2n)CO+(2n)H₂O=>(2n)CO₂+(2n)H₂

(n)CO+(2n)H₂=>CₙH₂ₙ₊₁OH+(n−1)H₂O

In a further embodiment of the present invention the processes of the first and the second embodiment may be combined so that a combination of liquid alkanes and alcohols are obtained from energy, H₂O and carbon dioxide.

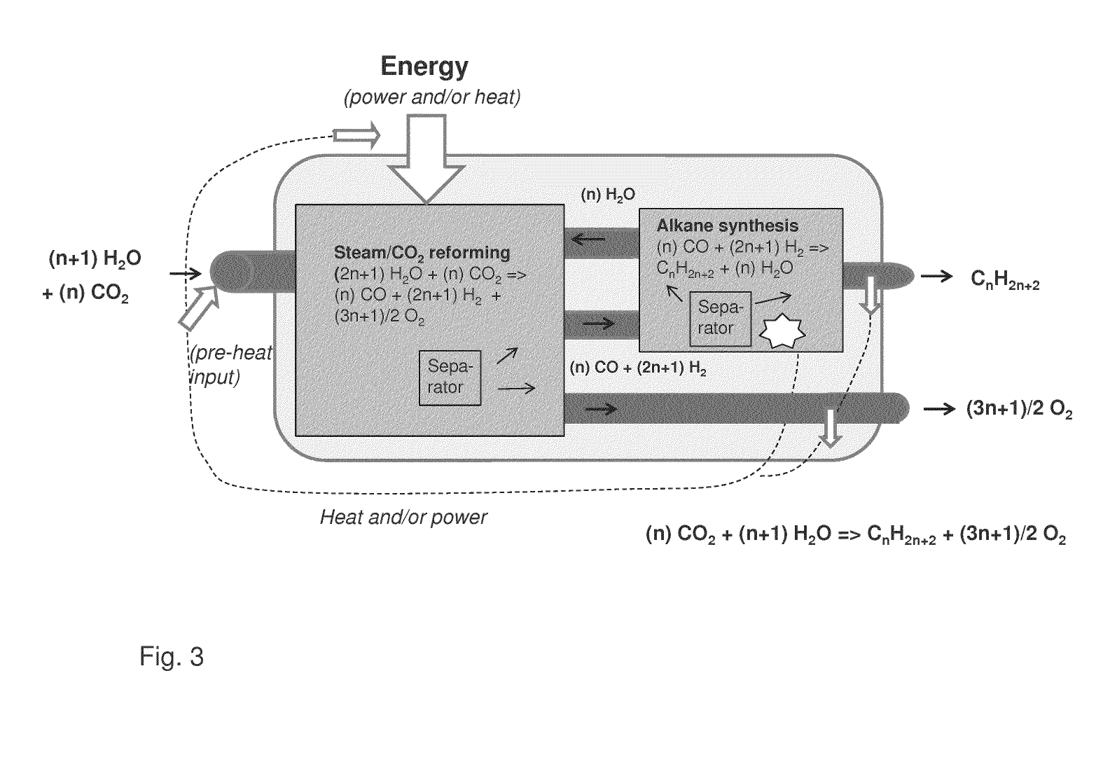

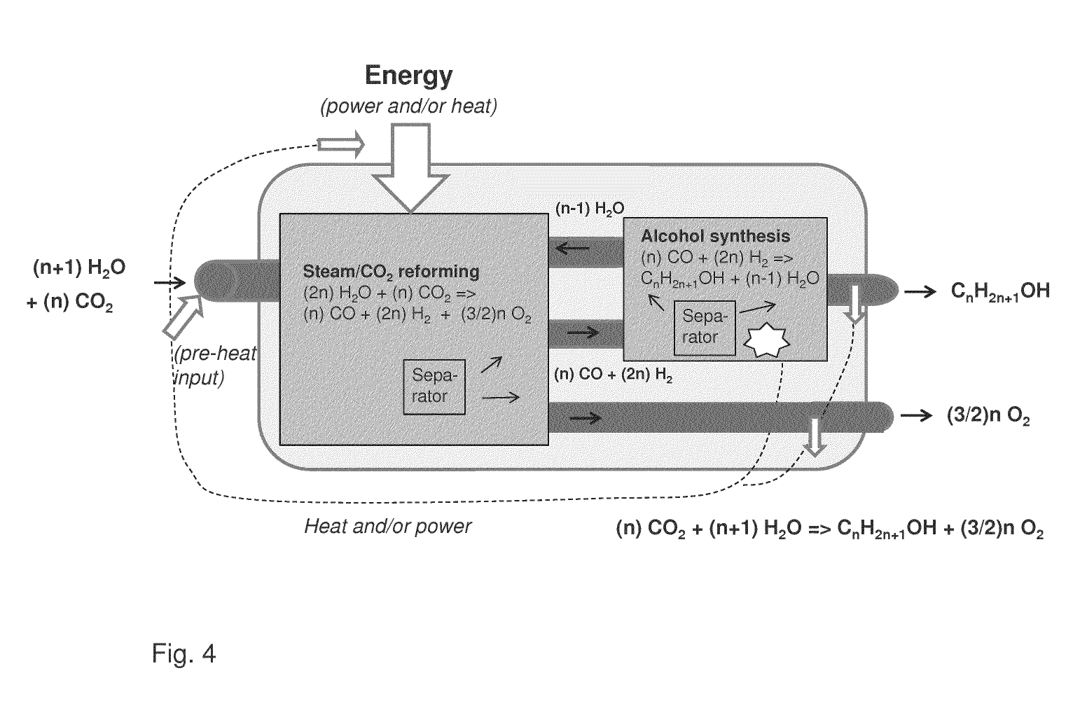

FIGS. 3 and 4 illustrate two further embodiments of the present invention comprising two reactions; steam/CO2 reforming and alkane or alcohol synthesis to produce alkane or alcohol.

The total reactions of embodiment 3 and 4:

Alkane Production (3):

(2n+1)H₂O+(n)CO₂>(n)CO+(2n+1)H₂+(3n+1)/2O₂

(n)CO+(2n+1)H₂=>CₙH₂ₙ₊₂+(n)H₂O

Alcohol Production (4):

(2n)H₂O+(n)CO₂=>(n)CO+(2n)H₂+(3/2)nO₂

(n)CO+(2n)H₂=>CₙH₂ₙ₊₁OH+(n−1)H₂O

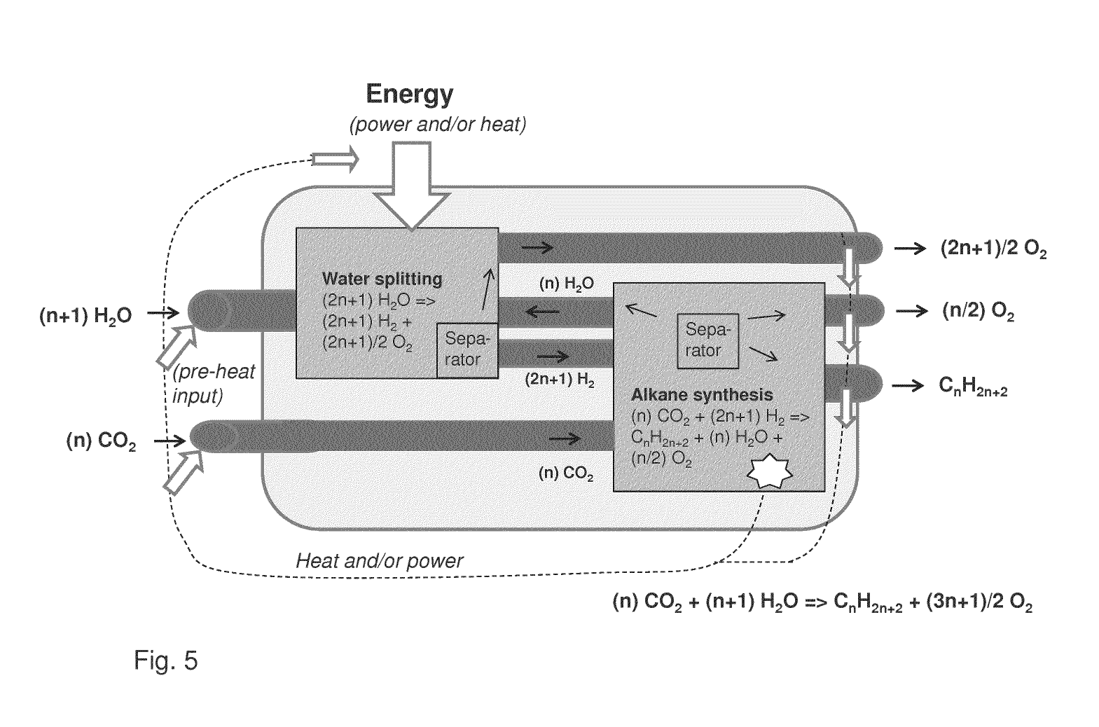

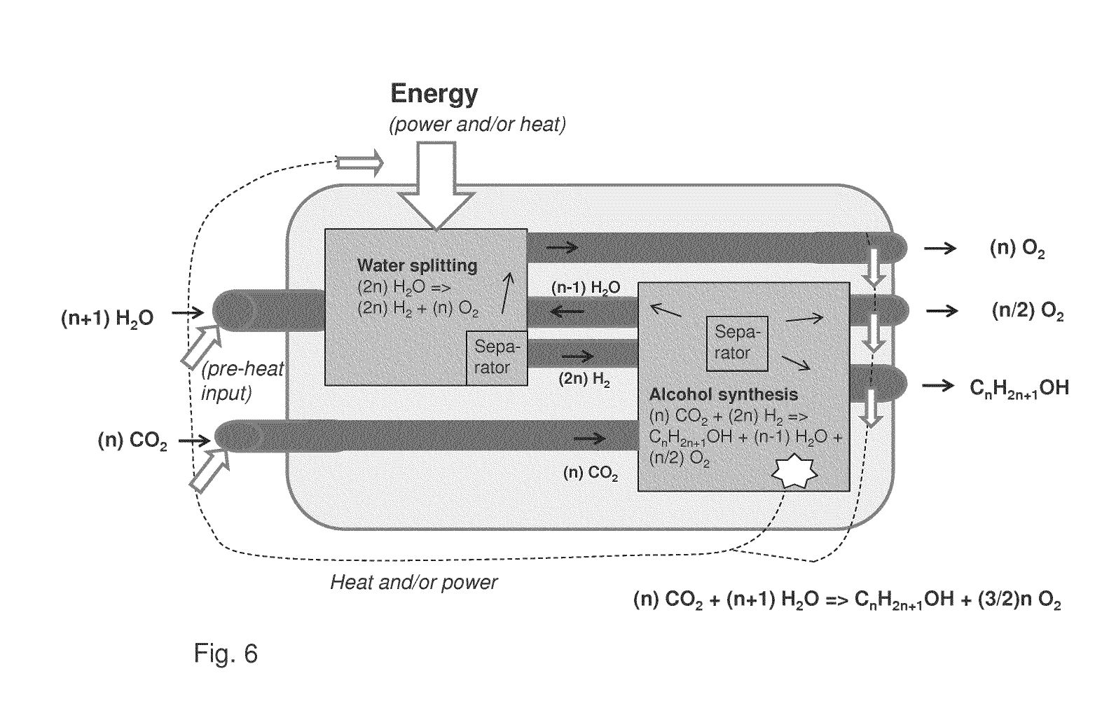

FIGS. 5 and 6 illustrate two further embodiments of the present invention comprising two reactions; water splitting and alkane or alcohol synthesis to produce alkane or alcohol.

The total reactions of embodiment 5 and 6:

Alkane Production (5):

(2n+1)H₂O=>(2n+1)H₂+(2n+1)/2O₂

(n)CO₂+(2n+1)H₂=>CₙH₂ₙ₊₂(n)H₂O+(n/2)O₂

Alcohol Production (6):

(2n)H₂O=>(2n)H₂+(n)O₂

(n)CO₂+(2n)H₂=>CₙH₂ₙ₊₁OH+(n−1)H₂O+(n/2)O₂

1. Energy uploading method transferring energy into liquid hydrocarbon comprising steps
a) preparing a mixture of hydrogen and carbon monoxide from carbon dioxide, H₂O and energy,
b) reacting said mixture to form liquid hydrocarbon,
c) transferring heat energy from the formed liquid hydrocarbon to the carbon dioxide and or the H₂O.

2. Energy uploading method according to claim 1, wherein step a) comprises decomposition of carbon dioxide into carbon monoxide and oxygen.

3. Energy uploading method according to claim 2, wherein step a) further comprises reacting a part of the carbon monoxide with H₂O to form carbon dioxide and hydrogen and transferring the formed carbon dioxide to the decomposition of carbon dioxide.

4. Energy uploading method according to claim 1, wherein step a) comprises combined steam reforming and carbon dioxide reforming.

5. Energy uploading method transferring energy into liquid hydrocarbon comprising steps
d) preparing hydrogen from H₂O and energy,
e) preparing a mixture of hydrogen and carbon dioxide,
f) reacting said mixture to form liquid hydrocarbon,
g) transferring heat energy from the formed liquid hydrocarbon to the carbon dioxide and or the H₂O.

6. Energy uploading method according to claim 1, wherein oxygen is produced as a by-product.

7. Energy uploading method according to claim 6, wherein the method comprises transferring heat from the oxygen to the carbon dioxide and or the H₂O.

8. Method according to claim 1, wherein the energy supplied is heat energy.

9. Method according to claim 1, wherein the energy supplied is sustainable energy.

10. Method according to claim 1, wherein the liquid hydrocarbon is alcohol CₙH₂ₙ₊₁OH, where n=1-20, preferably n=1-6.

11. Method according to claim 1, wherein the liquid hydrocarbon is alkane CₙH₂ₙ₊₂, where n=5-17, preferably n=5-10.

12. Method according to claim 1, wherein the step c) or g) comprises converting heat from exothermic reactions to power to be used in endothermic processes.
